(19) United States
(12) Patent Application Publication
(10) Pub. No.: US 2018/0042496 A1
(43) Pub. Date: Feb. 15, 2018

(54) SYSTEM AND METHOD FOR MEASURING VITAL SIGNS

(71) Applicant: Pelletric LLC, Fairfield, CT (US)

(72) Inventors: Shem B. Lachhman, Fairfield, CT (US); Joel S. Douglas, Bonita Springs, FL (US)

(21) Appl. No.: 15/728,540

(22) Filed: Oct. 10, 2017

Related U.S. Application Data
(63) Continuation-in-part of application No. 14/858,157, filed on Sep. 18, 2015.
(60) Provisional application No. 62/054,672, filed on Sep. 24, 2014.

(51) Int. Cl. A61B 5/0205; A61B 5/1455; A61B 5/11; A61B 5/00

(52) U.S. Cl. CPC A61B 5/02055; A61B 5/6803; A61B 5/14551; A61B 5/0008; A61B 5/0015; A61B 5/11; A61B 5/742; A61B 5/7271; A61B 5/021

(57) ABSTRACT

A portable wearable computing device configured to continuously obtain data indicative of a patient's vital signs is disclosed. The portable wearable computing device includes a temperature sensor configured to obtain data indicative of body temperature of the patient. The portable wearable computing device further includes a blood oxygen saturation sensor configured to obtain data indicative of amount of oxygen present in the patient's body. The portable wearable computing device further includes an arterial waveform sensor configured to obtain data indicative of an arterial waveform produced by the patient's artery. The portable wearable computing device further includes a processor coupled to the temperature sensor, the blood oxygen sensor, and the blood pressure sensor, and configured to receive the obtained data indicative of the patient's vital signs.

### CROSS-REFERENCE TO RELATED APPLICATION

This application claims priority from U.S. Patent Application No. 62/054672 filed on Sep. 24, 2014, which is incorporated by reference herein in its entirety. This application continuation in part of U.S. patent application Ser. No. 14/858,157 filed on Sep. 18, 2015, which is incorporated by reference herein in its entirety.

### FIELD OF DISCLOSURE

The present disclosure relates to the field of healthcare data measurement. More particularly, the present disclosure relates to a wearable device for measuring vital signs of a patient.

### BACKGROUND

High blood pressure or hypertension is one of several factors that can increase the risk of myocardial infarction (heart attack) and cerebrovascular accidents or stroke. Post-operative and post-myocardial infarction patients are required to closely monitor their blood pressure in order to prevent another cardiovascular failure which can lead to paralysis, mortality, or very high cost medical bills. Therefore, it is desirable and important to continuously monitor blood pressure in cardiovascular disease, diabetic, and obese patients.

Invasive systems and methods exist for measuring the blood pressure of a patient. For example, invasive blood pressure monitors typically utilize catheters with a pressure transducer or sensor on the tip. These devices are also known as intravascular pressure sensors. However, catheter blood pressure monitors require surgical implantation which can lead to infection requiring the patient to undergo another surgical procedure, which can extend the patient's stay in the medical clinic. In addition, the catheter method is also large in size and requires bulky and high cost external equipment, which may not be suitable for at-home continuous measurements. Implantable elastic cuffs with micro-electromechanical device (“MEMS”) pressure sensors are another form of an invasive device to measure blood pressure. The cuffs are surgically implanted around the blood vessel to measure the pressure change through the expansion of the walls. However, this system and method exposes the patient to a number of risks such as infection and collapsing of the cuff which can increase the chance of experiencing a heart attack or other cardiovascular-related complications. Furthermore, the implanted cuffs may also require several surgeries for performing maintenance of the system.

Noninvasive systems and methods also exist for measuring the blood pressure of a patient. In one example, cuffs or sphygmomanometers are placed either around the wrist or upper arm of the patient. Other methods include a blood pressure monitoring watch. However, these systems and methods do not provide the capability for continuous measurements to be transported to the primary physician. In addition, these systems and methods can also create great inconvenience and momentary discomfort for the patient. Also, the sphygmomanometer is error prone. In particular, the size of a sphygmomanometer must be correctly adjusted to give an accurate blood pressure reading. An adjustment where the cuff is too tight can produce a higher reading while a loose adjustment can produce a lower reading.

### SUMMARY

In one example, a portable wearable computing device configured to continuously obtain data indicative of a patient's vital signs is disclosed. The portable wearable computing device is formed in the shape of an ear bud and includes a temperature sensor configured to obtain data indicative of body temperature of the patient. The temperature sensor is positioned in the ear so that it can rotate to allow for accurate measurement of the temperature at the tympanic membrane. The portable wearable computing device further includes a Blood Oxygen Saturation (BOS) sensor configured to obtain data indicative of the amount of oxygen present in the patient's body. The portable wearable computing device further includes an arterial waveform sensor configured to obtain data indicative of an arterial waveform produced by the patient's artery. The portable wearable computing device further includes a processor coupled to the temperature sensor, the blood oxygen sensor, and the blood pressure sensor, and configured to receive the obtained data indicative of the patient's vital signs from the patient's body and more specifically from the region of the ear.

In one example, a method for continuously obtaining vital sign data is disclosed. The method includes the step of disposing a wearable measurement device on a patient's body and more specifically from the region of the ear.

The method further includes the step of continuously acquiring data representative of the patient's vital signs from the wearable measurement device. The method further includes the step of converting the acquired data in real time. The method further includes the step of communicating the converted data.

In one example, a non-invasive system for continuously monitoring blood pressure of a patient is disclosed. The system includes a sensor disposed on the patient and in communication with the superficial temporal artery. The Temporoparietal Fascia contains the superficial temporal artery. The sensor is configured to acquire data indicative of an arterial waveform from the patient and to wirelessly communicate the acquired data indicative of the arterial waveform to a patient computer. The patient computer is configured to receive the communicated data indicative of the arterial waveform and to derive systolic and diastolic blood pressure data based on the received data representative of the arterial waveform.

In one exemplary example, due to the nature of the anatomy of the face a means of accurately determining the blood pressure and pulse oximetry is needed that cancels the effects of temperature on the skin and vascular plexus that the temporal artery is part of.

In one exemplary example, due to differing facial structures there is a need to insure that the blood pressure sensor is aligned with the temporal artery both axial compliance and radial adjustment. The radially adjustment is with respect to the ear and the sensor must be capable of applying adequate pressure to the temporal artery so an accurate reading can be determined. Additionally, due to variability of the skin and capillaries under the skin the ambient temperature needs to be accounted to allow for an accurate blood pressure measurement.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for measuring vital signs.

FIG. 2 is an example arterial waveform.

FIG. 3 is an example wearable measurement device.

FIG. 4 is an example wearable measurement device.

FIG. 5 is an example wearable measurement device.

FIG. 6 is an example wearable measurement device.

FIG. 7 is an example wearable measurement device.

FIG. 8 is an example wearable measurement device.

FIG. 9 is an example wearable measurement device.

FIG. 10 is an example wearable measurement device.

FIG. 11 is a schematic illustrating an example positioning of an example wearable measurement device.

FIG. 12 illustrates an example system for measuring vital signs.

FIG. 13A illustrates an example blood pressure monitoring patch.

FIG. 13B illustrates an example blood pressure monitoring patch.

FIG. 13C illustrates an example blood pressure monitoring patch.

FIG. 13D illustrates an example blood pressure monitoring patch.

FIG. 14 illustrates an example absorption mode BOS patch.

FIG. 15 is an example reflectance mode BOS patch.

FIG. 16A is an example wearable measurement device in unfolded position.

FIG. 16B is an example wearable measurement device in folded position.

FIG. 16C is an example wearable measurement device in unfolded position.

FIG. 16D is an example wearable measurement device in folded position.

FIG. 16E is an example wearable measurement device in unfolded position.

FIG. 16F is an example wearable measurement device in unfolded extended position.

FIG. 17A and FIG. 17B is an example wearable measurement device.

FIG. 18 illustrates an example method for measuring vital signs.

FIG. 19 is a block diagram of an example computer for implementing an example third-party computing device of FIG. 1.

FIG. 20A and FIG. 20B is the design of the blood pressure sensor arm showing both axial compliance and radial adjustment.

FIG. 21 shows the superficial temporal artery relatively surface of the skin.

FIG. 22A shows the uncorrected curve for an ambient temperature of 26 degrees C. versus the corrected curve when the correction factor is applied.

FIG. 22B shows the curve taken with a conventional blood pressure cuff.

### DETAILED DESCRIPTION

An ear bud is defined as a device of the invention used to continuously monitoring vital signs of a patient such as blood pressure, Blood Oxygen Saturation (BOS), heart rate, body temperature, respiratory rate, and body position of the patient from the region of the patient's ear.

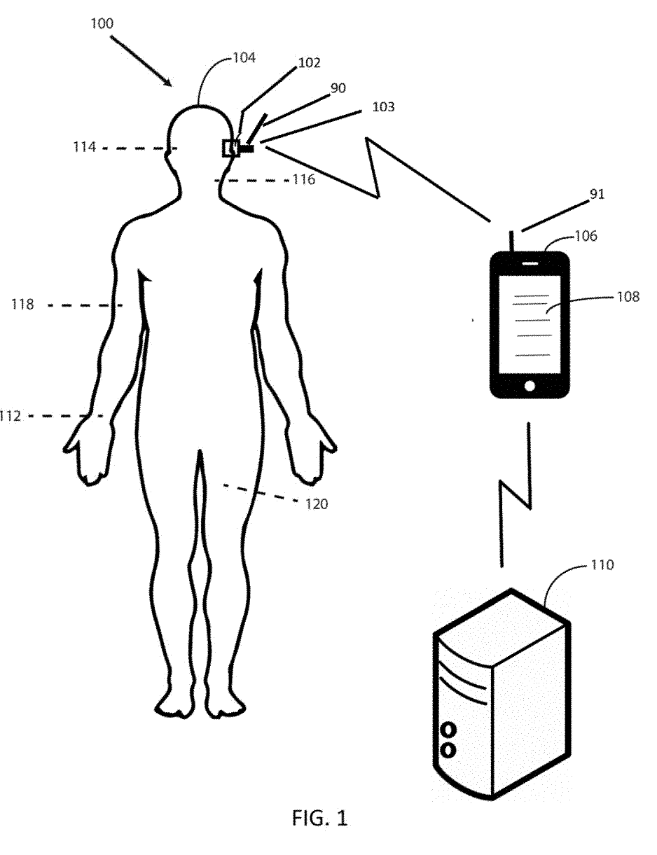

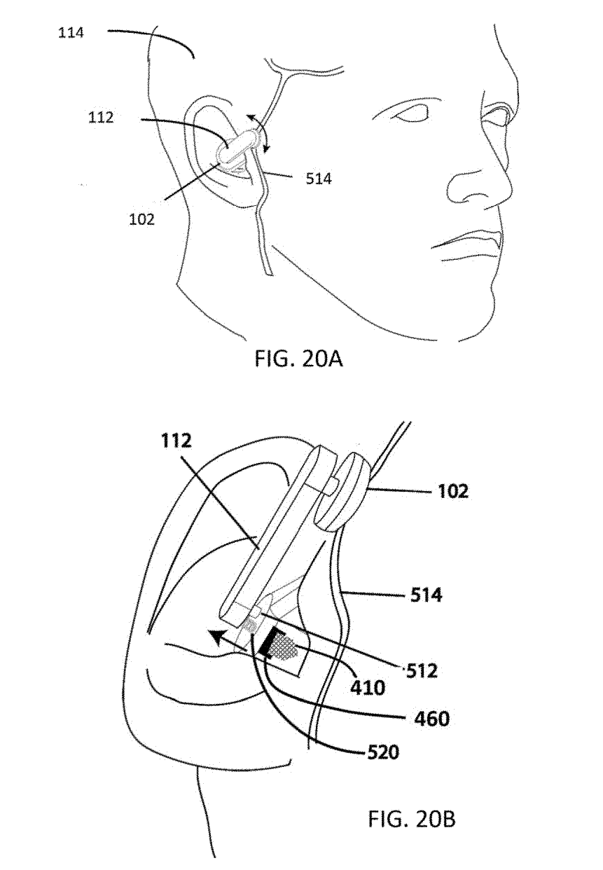

Referring to FIG. 1 and FIG. 20A, FIG. 1 illustrates an example system 100 for measuring vital signs. It should be appreciated that the system 100 may be utilized in a hospital setting or in everyday living. For example, system 100 may be utilized by a health care professional to measure vital signs of a patient 104, either during a clinical visit or at a bedside while admitted at a hospital. In another example, the patient 104 may use system 100 to measure own vital signs while at home or at any other convenient location. System 100 includes a wearable measurement device 102 that has processor 103 for continuously monitoring vital signs such as blood pressure, Blood Oxygen Saturation (BOS), heart rate, body temperature, respiratory rate, and body position of the patient 104 and these sensors are in communication with processor 103 which is part of wearable measurement device 102. Processor 103 can be configured to interpret the data such as vital signs such as blood pressure, blood oxygen saturation, heart rate, body temperature, respiratory rate, and body position. It should be appreciated that wearable measurement device 102 may include any suitable wearable device, such as a ear bud, watch, a bracelet, a ring, an earring, and so on, that may incorporate suitable computer hardware and software components for collecting vital signs.

The wearable measurement device 102 is non-invasive that is configured to come in direct contact with the patient's 104 skin, without requiring a surgical procedure. It should be understood that although the example system 100 depicts the wearable measurement device 102 positioned on the patient's 104 ear the wearable measurement device 102 may be positioned on any portion of the patient's 104 body suitable for continuously monitoring vital signs. For example, the wearable measurement device 102 may be positioned near the superficial temporal artery 514 of the head 114. In other examples, the wearable measurement device 102 may be positioned near the carotid artery in the neck 116, the brachial artery in the arm 118, or the femoral artery in the leg 120.

The wearable measurement device 102 includes a processor 103 and a plurality of sensors (not shown) working to obtain vital sign information. In one example, each sensor may gather more than one type of information. The sensors can be semiconductor sensors or optical sensors, for example. In addition, the wearable measurement device 102 includes a low power circuitry, an integrated power supply, application-specific integrated circuits, and a housing that attaches to the surface of the skin of the patient 104 without requiring a surgical procedure. The wearable measurement device 102 further includes a wireless transmission antenna 90 in communication with processor 103 such as Wi/Fi, Bluetooth or Near Field Communications antenna for wirelessly communicating the obtained vital sign information.

The system 100 further includes a patient computing device 106 having a wireless antenna 91 for receiving the vital sign information from the wearable measurement device processor 103 and a user interface 108 for displaying the received information in a concise, organized fashion. The patient computing device 106 may be any suitable device such as a smart phone, a tablet, a personal computer, or a smart watch. The patient computing device 106 includes computer readable tangible storage device and a software application for processing received information and converting the information into common parameters such as systolic blood pressure or diastolic blood pressure before displaying the information on the user interface 108. The computer readable tangible storage device can be selected from different memory options such as solid-state drive (SSD), Random Access Memory, disk drive or tape drive device. Alternatively, processor 103 can be configured to interpret the data such as vital signs from the sensors such as blood pressure, blood oxygen saturation, heart rate, body temperature, respiratory rate, and body position.

In one example, the software application of the patient computing device 106 also communicates information to the wearable measurement device 102 using processor 103. For example, the computing device 106 may be configured to receive information about operational settings or parameters and to communicate the information to the wearable measurement device 102. In one example, the wearable measurement device 102 may send using processor 103 other suitable data to the patient computing device, other than vital sign data. For example, the wearable measurement device 102 may communicate information using processor 103 such as battery fife, improper measurement alerts, indications of misaligned sensors, connectivity problems, and so on.

In one example, the obtained vital sign information is also communicated to a third-party computing device 110 such as a device associated with, a physician, a family member, or a third-party data-monitoring service. The third-party computing device 110 may be any suitable device such as a smart phone, a tablet, a personal computer, a computer server, or a smart watch, for example. In one example, the third-party computing device 110 includes a computer readable tangible storage device and an electronic health records (“EHR”) system that stores patient health records and is configured to store the received vital sign information in association with the patient's 104 health records. The computer readable tangible storage device can be selected from different memory options such as solid-state drive (SSD), Random Access Memory, disk drive or tape drive device. In one example, the patient computing device 106 is configured to automatically communicate all received vital sign information to the third-party computing device 110. In another example, the patient computing device 106 is configured to communicate the received vital sign information to the third-party computing device 110 or an alert only when the vital signs are outside of a normal measurement range. Accordingly, a patient's 104 physician or family member may be automatically notified when the patient's blood pressure is high, for example. In one example, the wearable measurement device 102 may be configured to communicate directly with the third-party computing device 110. Alternatively, processor 103 can be configured to interpret the data such as vital signs such as blood pressure, blood oxygen saturation, heart rate, body temperature, respiratory rate, and body position.

In one example, before the wearable measurement device 102 using processor 103 can begin to stream vital sign information to the third-party computing device 110, the wearable measurement device 102 using processor 103 performs a digital handshake with the third-party computing device 110. For example, the wearable measurement device 102 using processor 103 may communicate a unique identification number or other suitable identifying information for the third-party computing device 110 to confirm the identity of the wearable measurement device 102 and the associated patient 104. In particular, after a wearable measurement device 102, including a unique serial number is assigned to a patient 104, the unique serial number is provided to the third-party computing device 110 by the wearable measurement device 102 using processor 103. The third-party computing device 110 may then be configured to associate with a specific patient record of patient 104 all vital sign information received from the wearable measurement device 102 using processor 103 having the unique serial number.

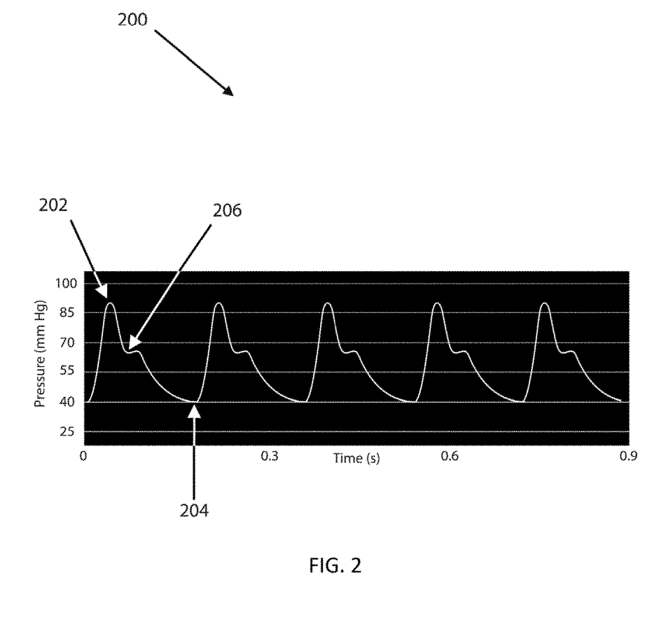

In order to non-invasively monitor blood pressure with minimal interference from artifacts such as movements from walking, coughing or sneezing, system 100 monitors' vibrations exhibited from arterial palpation. Arterial palpation is a result of constant contraction and expansion of the arterial walls to pump or carry blood to extremities within the human body. Several major arteries exhibit throbbing or palpation, that can be felt through the skin. By monitoring the palpation of an artery, system 100 is able to acquire an arterial waveform, from which systolic and diastolic blood pressure readings can be derived. FIG. 2 illustrates an example arterial waveform 200 acquired from monitoring an arterial palpation, including systolic 202 and diastolic 204 blood pressure readings and a dicrotic notch 206.

Palpations from arterial wall expansion and contraction can be found at any of the carotid artery, superficial temporal artery, femoral artery, or radial artery, for example. Since the cardiovascular system is a closed-looped system, the pulse at different locations on the body will remain the same.

FIG. 3 illustrates an example wearable measurement device 300 designed to be positioned at a patient's ear, near the superficial temporal artery. The example wearable measurement device 300 may be configured to be secured to the ear such that the device remains in proximate position to the superficial temporal artery even when the patient performs moderate movements. In one example, the wearable measurement device 300 may be secured to the patient's ear in such a way as to prevent the device from slipping off or out of place even when the patient performs exercise movements such as jogging.

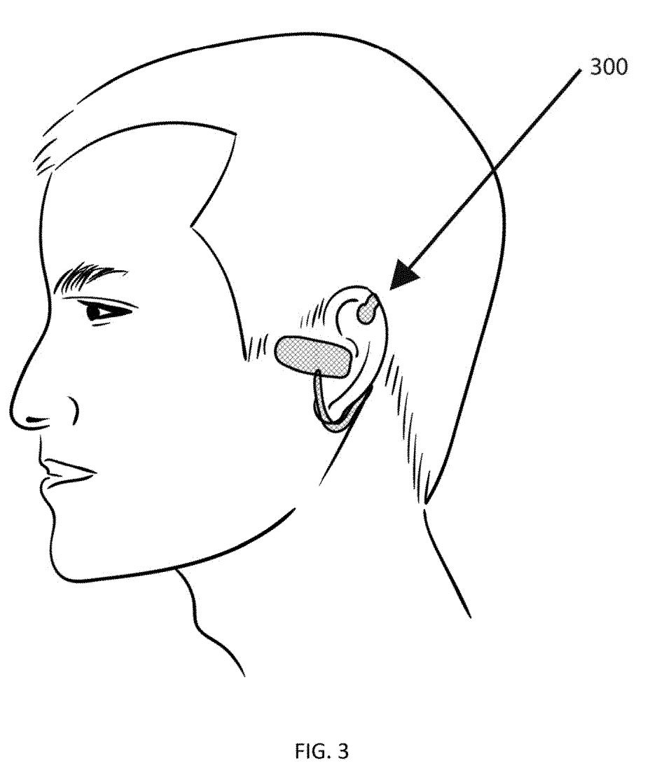

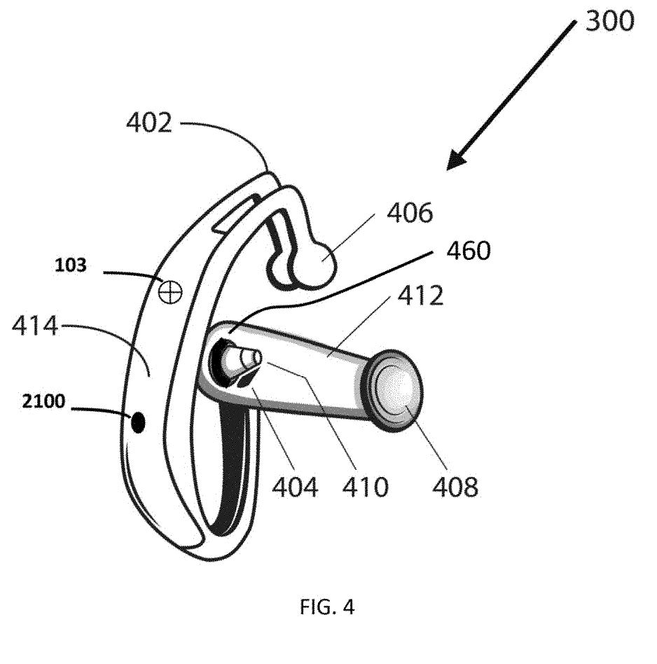

Referring to FIG. 4 and FIG. 20A, FIG. 4 illustrates an example wearable measurement device 300. The wearable measurement device 300 includes features that facilitate vital sign data collection as well as that facilitate positioning on the ear of a patient. For example, the wearable measurement device 300 includes, using processor 103, an ear clip 402 that holds on to the top of the ear at the helix. In one example, the wearable measurement device 300 further includes a tragus clip 410 to clip onto the tragus of the ear and to further facilitate a secure positioning of the device on the ear.

To facilitate vital sign data collection, the wearable measurement device 300 includes a sensor 406 in communication with processor 103 configured to measure blood oxygen saturation. The wearable measurement device 300 further includes a sensor 408 in communication with processor 103 configured to measure or acquire an arterial wave form which can then be translated into heart rate and blood pressure. The wearable measurement device 300 further includes a sensor 410 in communication with processor 103 configured to measure body temperature. The wearable measurement device 300 further includes a housing 412 for storing additional suitable electronics, such as a processor 103 for executing suitable program instructions associated with the described functionality of the wearable measurement device, or sensors, such as an accelerometer and a gyroscope for measuring a patient's body position. In one example, the housing 412 is adjustable to allow for movement and proper alignment of sensor 408 with a patient's superficial temporal artery. For example, the housing may be configured to extend and retract in order to properly fit a patient's ear. It should be appreciated that other suitable portions of the wearable measurement device 300 may be adjustable to allow for proper fit on a patient's ear. For example, the body 414 of the wearable measurement device 300 may be adjustable to properly fit around the back of the ear. In addition the wearable measurement device 300 includes an ambient temperature sensor 2100 which is used to take the ambient temperature.

In one example, the sensor 410 is configured to be placed inside an ear canal of a patient's ear to measure the body temperature. Rotatable hub 460 allows the sensor 410 to align in the ear canal so that the sensor is positioned to read the temperature at the tympanic membrane inside the ear and the rotatable hub 460 also provides a spring load force that pushes the sensor 410 into the ear canal. It should be appreciated that the sensor 410 is configured to fit inside an ear canal of various sizes. In one example, the sensor 410 is an optical sensor, such as a thermopile or IR sensor, configured to measure temperature.

It should be appreciated that wearable measurement device 300 illustrated is one example of a possible configuration and that processor 103, blood oxygen saturation sensor 406, the arterial wave form sensor 408, and the body temperature sensor 410 may be positioned on the device in any suitable configuration. In addition, the wearable measurement device 300 may be configured to be secured to any suitable portion of a patient's body. Examples of a blood oxygen saturation sensor 406, an arterial wave form sensor 408, and a body temperature sensor 410 will now be described in more detail.

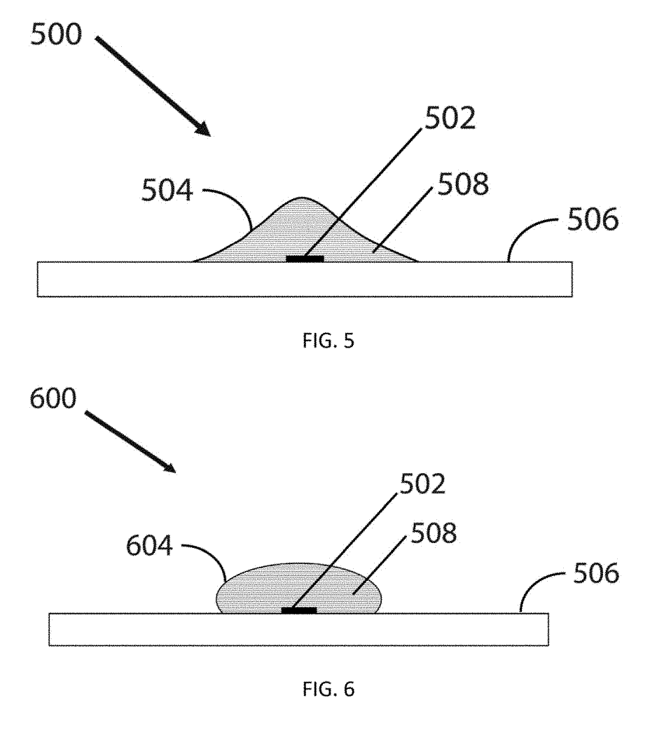

FIG. 5 illustrates an example wearable measurement device 500 including a sensor configured to acquire an arterial waveform 200. In particular, in order to acquire the arterial waveform 200 and derive the systolic and diastolic blood pressure accurately, a wearable measurement device 500 includes a high sensitivity pressure sensor 502 that is placed over the location of a palpation and anchored to the skin. High sensitivity pressure sensors are low in cost, simple to integrate with other electronics, and can measure small changes in pressure, making it suitable for this application. Sensor 502 can be selected from the following sensors Honeywell TBP Board Mount Pressure Sensors TBPMANN150PGUCV, Amphenol NPC Nova Sensors NPC-100, and STMicroelectronics MEMS Pressure Sensor LPS33HW or similar commercially available devices. Translation of the pressure changes within the arterial walls to the pressure sensor 502 with accuracy is facilitated by a flexible protective layer 504 in the shape of a mound structure that allows the artery to be lightly compressed against the bone. This technique is commonly used when checking a pulse. The palpation from the arterial pulse can be felt with trained fingertips by compressing the artery against the bone and can only be felt in areas where the arteries are able to be compressed against a reference bone. The sensor 502 is placed inside of the flexible protective layer 504 and anchored to a rigid substrate 506. In one example, the mound formed by the flexible protective layer 504 is filled with a low viscosity material 508 and anchored to the substrate 506. In one example, as illustrated in FIG. 6, a flexible protective layer 604 forms a bubble instead of a mound 504.

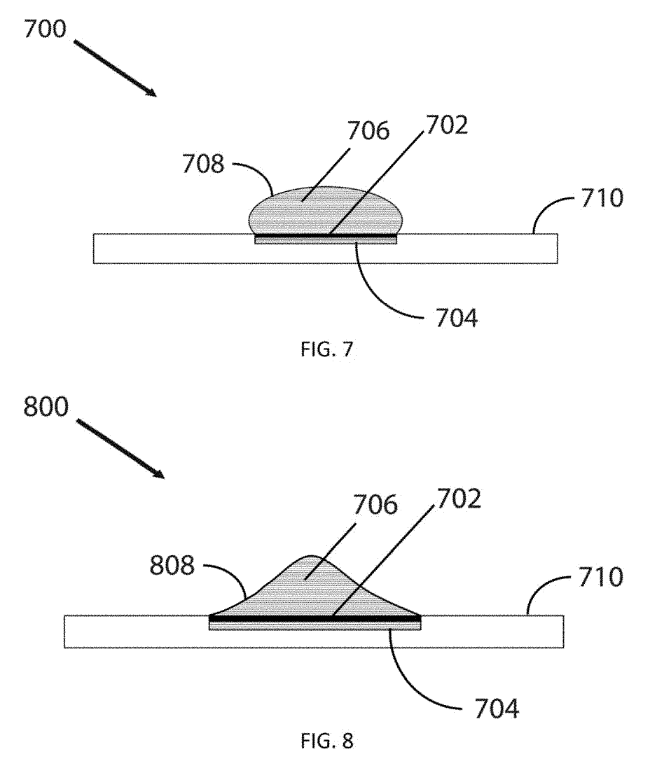

FIG. 7 illustrates another example wearable measurement device 700. In an alternative to using a semiconductor sensor, the wearable measurement device 700 incudes a piezoelectric thin film sensor 702 which exhibits high sensitivity to vibration and mechanical forces such as bending. Piezoelectric films are typically 10 to 150 microns in thickness. They offer several advantages such as low cost, simple signal conditioning, low noise, low power consumption, and high sensitivity. Sensor 702 can be selected from the following commercially available devices such as TE Connectivity, Piezo Film Sheets, CAT-PFS0003, and Alpha, Force Sensors, MF02-N-221-A01 or similar commercially available devices. The piezoelectric film 702 is placed over a cavity 704 filled with low viscosity material 706 and capped with a flexible protective layer 708 in the shape of a bubble structure and anchored to a substrate 710. Placing the piezoelectric thin film 702 over the cavity 704 will enable the piezoelectric thin film 702 to vibrate, and bend when an outside force pushes against the protective layer 708. In one example, as illustrated in FIG. 8, a flexible protective layer 808 forms a mound instead of a bubble.

Referring back to FIG. 1, the wearable measurement device 102 may be configured to measure blood oxygen saturation. Blood oxygen saturation (SOS) is a relative measure of the amount of oxygen in the blood. A typical measurement will normally occur on the index finger, the ear, and other parts of the body where the flesh is thin. In one example, the wearable measurement device 102 utilizes an optical method to measure BOS on the earlobe or pinna (top portion of the ear) of the patient 104. This location is used to negate the device 102 from movement and physical abrasion. It will also work in conjunction with the blood pressure monitoring portion of the device 102 to gather multiple vital signs.

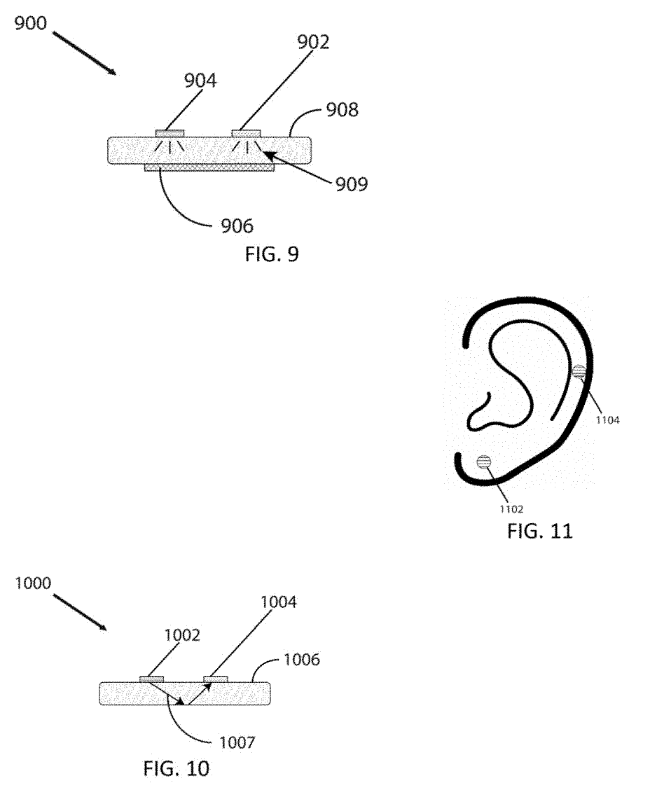

FIG. 9 illustrates another example wearable measurement device 900 configured to use an absorption mode to measure blood oxygen saturation. The wearable measurement device 900 includes an infrared LED light source 902 with wavelength of 940 rim, a red LED light source 904 with operating wavelength of 660 rim, and a photo-detector 906 to measure the blood-oxygen saturation of the user. In particular, the LED light sources 902 and 904 are placed on one side of the ear lobe 908, and the photo-detector 906 is placed on the opposing side and aligned to the LED light sources 902 and 904. The red LED 904 and the infrared LED 902 blink multiple times independently while the photo-detector 906 measures the absorbed light passing through the ear. Photodiodes in the photo-detector 906 measure the changing absorbance at each of the wavelengths, allowing for determination of absorbance due to the pulsing arterial blood alone, excluding venous blood, skin, bone, muscle, fat, and so on.

FIG. 10 illustrates another example wearable measurement device 1000 configured to use a reflectance mode to measure blood oxygen saturation. Wearable measurement device 1000 includes both the LEDs 1002 and a photo-detector 1004 on a single side of the ear lobe 1006. In both examples illustrated in FIGS. 9 and 10, amplification of the signal is achieved through operational amplifiers and linear circuits such as high pass and low pass circuits. Operation of the example wearable measurement devices 900 and 1000 requires the LEDs 902, 904, and 1002 to flash, one at a time, respectively, while the photo-detectors 906 and 1004 measure the absorbed or reflected light respectively by measuring, oxygenated and deoxygenated hemoglobin present in the blood of the patient 102. Hemoglobin is a protein found within red blood cells that transports or carries oxygen. FIG. 11 is a schematic illustrating an example positioning of a wearable measurement device 900 or 1000 at either the ear lobe 1102 or the pinna 1104.

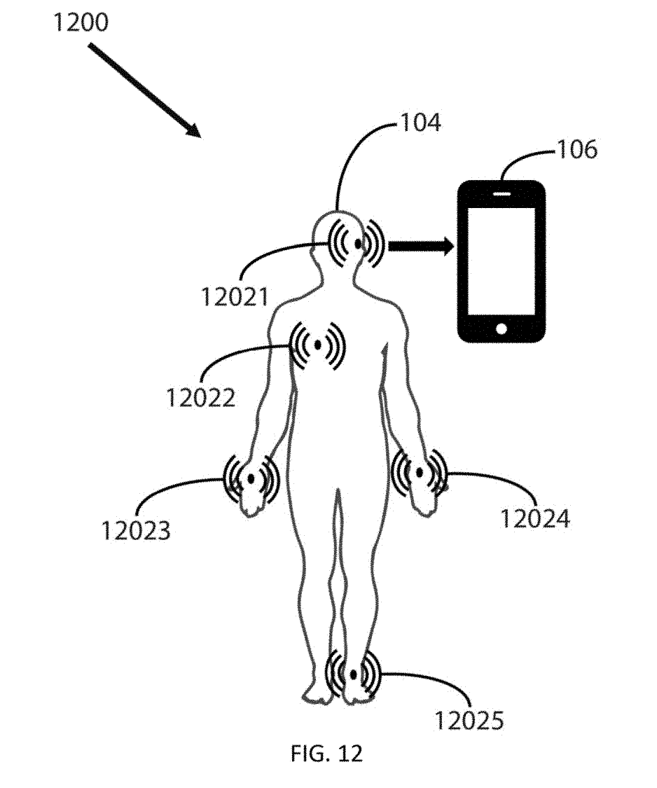

It should be appreciated that, although the example system 100 of FIG. 1 illustrates a single wearable measurement device 102, another example system 1200 may include a plurality of wearable measurement devices 12021, 12022, 12023, 12024 and 12025, as illustrated in FIG. 12. The network of wearable measurement devices 12021, 12022, 12023, 12024 and 12025, or flexible patches, gather multiple vital signs simultaneously and transmit the acquired information to a single patient computing device 106. Each flexible patch 12021, 12022, 12023, 12024 and 12025 locations on the patient 104 can be referred to as a node where each node may contain low power electronic circuits, a powering source and a module/antenna to transmit information wirelessly.

Blood pressure monitoring (“BPM”) nodes are placed over the artery where palpation can be found as discussed above while blood oxygen saturation (“BOS”) nodes are placed at the lobe or pinna of the ear. The BPM patch will incorporate the characteristics described in FIGS. 5-8 such as a pressure sensor or piezoelectric thin film housed within a bubble/mound-like structure and filled with low viscosity material while BOS patches contain the characteristics described in FIGS. 9-10.

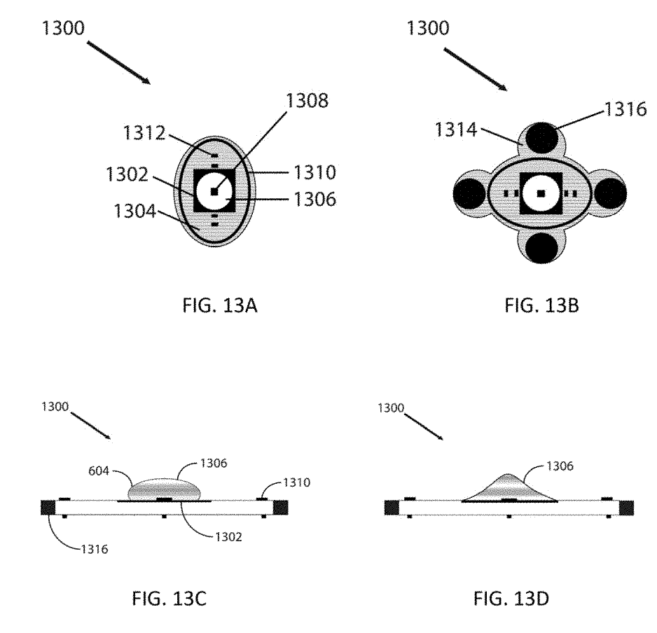

FIG. 13A illustrates an example BPM patch 1300. The BPM patch 1300 may include a rigid backing 1302 as a mechanical support structure disposed on a flexible substrate 1304 that will allow the compression of the artery by the mound/bubble protective layer 1306 disposed over the sensor 1308. BPM nodes 1300 are equipped with an adhesive ring 1310 along the edge. The flexible substrate may further incorporate additional suitable electronic components 1312. As illustrated in the aerial view of FIG. 13B, BPM patches 1300 can also incorporate flaps 1314 with adhesive material 1316 to further ensure movement of the patch 1300 is reduced to a minimum.

FIGS. 13C-13D illustrate side views of the example BPM patch 1300, including a bubble protective layer 1306 and a mound protective layer, respectively.

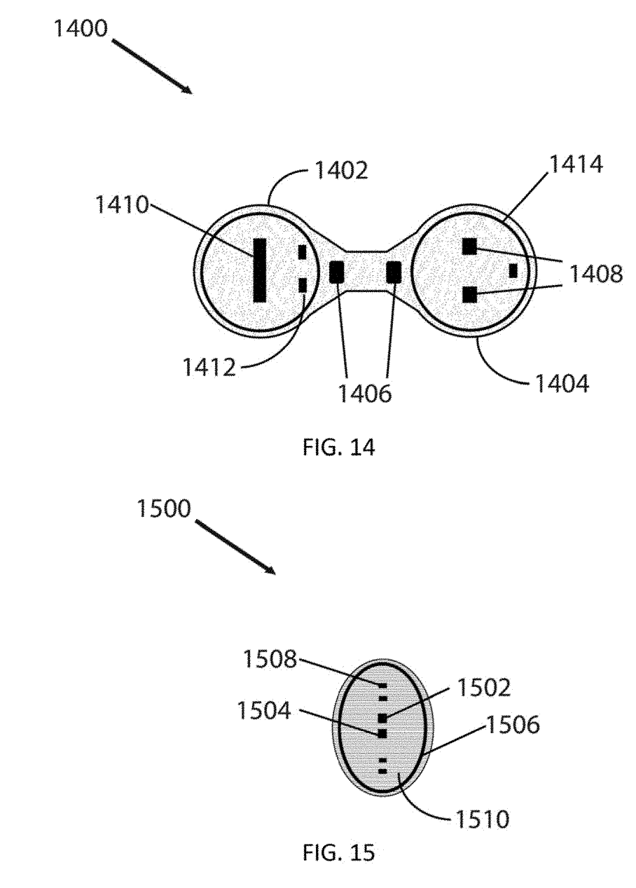

FIG. 14 illustrates an example absorption mode BOS patch 1400. The patch 1400 is configured to fold over an ear, or other suitable part of the body, where a first side 1402 is secured to either the front or the back of an ear and the second side 1404 is secured to the opposite side of the ear. Absorption mode BOS patches 1400 contain alignment markers 1406 to assist in aligning the first side 1402 with the second side 1404 when folding over and securing to an ear since misalignment of the first side 1402 and the second side 1404 can cause a misreading. The alignment markers 1406 can be in the form of a small dot or stud to assist in alignment. The absorption mode BOS patch 1400 further includes LEDs 1408 placed on one side of the ear and a photodiode 1410 on the other. The LED light 1408 passes through the ear and reaches the photodiode 1410, assisted by the alignment markers 1406, in order to capture an accurate measurement. Additionally, other suitable electronics 1412 may be embedded in the patch 1400. The absorption mode BOS patch 1400 further includes an adhesive 1414 for securing to an ear and to prevent movement. It should be appreciated that the patch 1400 may be fabricated from suitable flexible material.

FIG. 15 illustrates an example reflectance mode BOS patch 1500. The patch 1500 is configured to be secured to a flat, or relatively flat, portion of a body, rather than being folded over a portion of the body as the absorption mode BOS patch 1400 of FIG. 14 is configured to do. Similar to the absorption mode BOS patch 1400, the reflectance mode BOS patch 1500 also includes LEDs 1502, a photodiode 1504, an adhesive 1506, and other suitable electronics 1508 mounted to a flexible substrate 1510. However, the LEDs 1502 and the photodiode 1504 are disposed adjacent to one another, rather than on opposite sides.

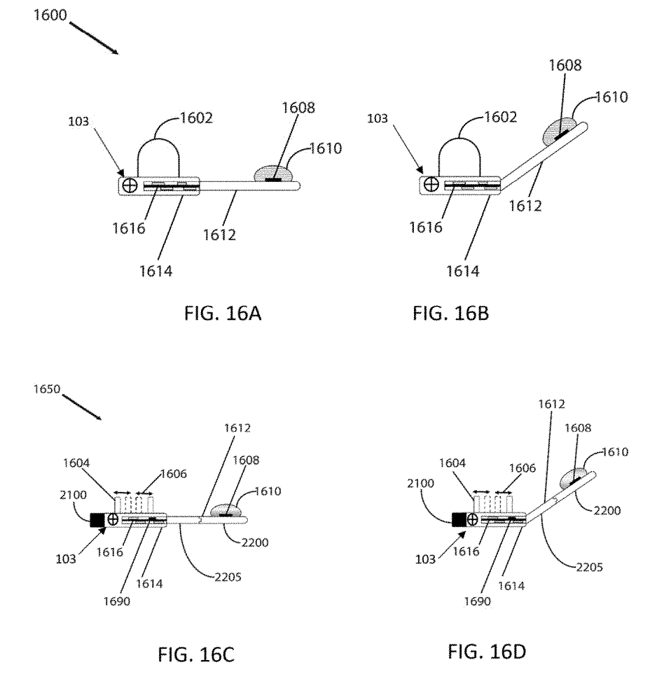

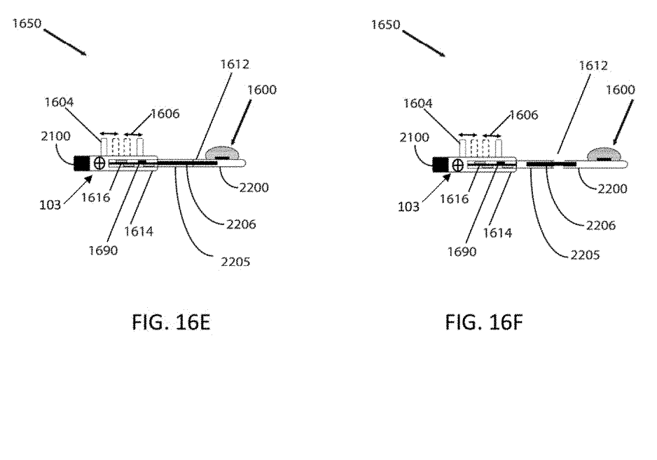

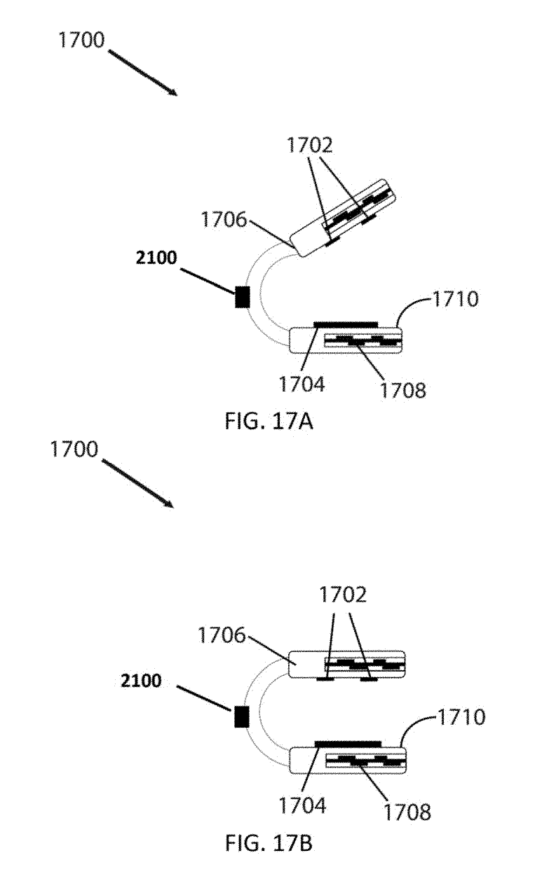

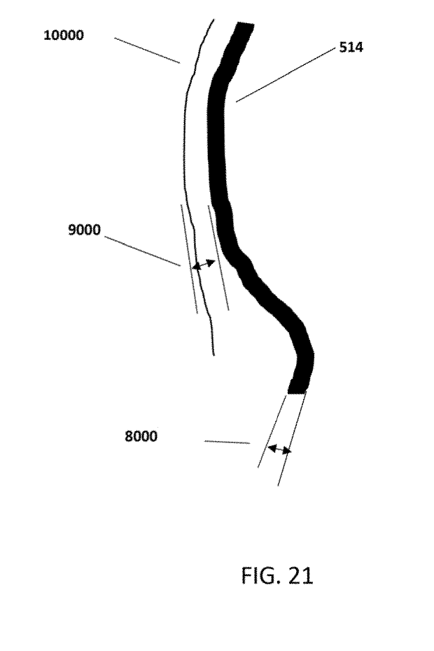

Using FIG. 4, FIG. 16A, FIG. 16B, FIG. 16C, FIG. 16D, FIG. 16E, FIG. 16F, FIG. 17, and FIG. 21, a wearable measurement device 300 includes independent components, a BPM 1600, a BOS 1700, an ambient temperature 2100 sensor and a body temperature sensor 1602 that can be used in combination or independently of one another. The BPM 1600, the BCS 1700, an ambient temperature 2100 sensor and a body temperature sensor 1602 each may include several sensors and their own electronics or power source. The ambient temperature 2100 sensor and a body temperature sensor 1602 gather information independently but transfer the information to the same patient computing device. The placement of the BPM sensor 1600 relative to the superficial temporal artery 514 allows the sensor to receive motion data which is directly related to the pulse within the superficial temporal artery. The anatomy of the face is such that the Temporoparietal Fascia contains the superficial temporal artery 514. The relatively small distance represented by Delta Y 9000 approximately 5-8 mm makes surface characteristics of the skin 10000 critical to the accuracy of the measurement. The change in ambient temperature has been shown to result in the superficial temporal artery diameter 8000 to vary as well as the Delta Y 9000 dimension. During, testing we found that as ambient temperature went up above 34 degrees C./93 degrees F. the intensity of the pulse went up and the wavelength was increased. This was mirrored as the ambient temperature went down below 28 degrees C./82 degrees F. the intensity of the pulse goes down and the wavelength was decreased, due to the well-known fact that capillaries contract when they are subjected to cold and increase with warmer conditions. As discussed in Moor Instruments Ltd “Microvascular Responses to Skin Heating: an Introduction Issue 1”. When the skin is heated, skin blood vessels dilate, more blood flows and we can detect this increase with laser Doppler. The increase in blood vessel diameter is not the only way in which blood flow increases; under normal, moderate room temperatures (20-24□C.), not all of the smallest vessels (capillaries) have blood flowing through them. Blood flow also increases by more of the capillaries allowing blood to flow through them (capillary recruitment).

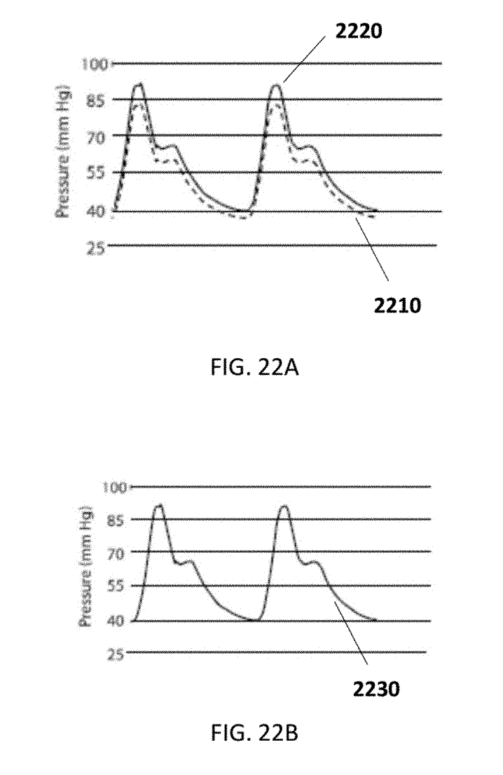

The variation in BPM readings caused by ambient temperature meant that we had to provide an algorithm that compensated for these small changes so that the accuracy of our readings would be consistent with the accuracy of the more significant reading taken from the bicep area of the body. During testing, we found that the readings varied by as much as 0.1% percent lower when the ambient temperature was above 34 degrees C./93 degrees F. The variation depended on the age of the person and varied between −0.02% to −0.1%. The readings varied by as much as 0.1% percent higher when the ambient temperature was below 28 degrees C./82 degrees F. The variation depended on the age of the person and varied between +0.03% to +0.1%. This compensation based on the data required that the data be modified to give accurate results based on the ambient temperature. As shown in FIG. 22A curve 2210 is the uncorrected curve for an ambient temperature of 26 degrees C. and curve 2220 is the resulting corrected curve when the correction factor is applied. As shown in FIG. 22B shows the curve 2230 taken with a conventional blood pressure cuff. FIG. 16A illustrates an example BPM component of a wearable measurement device 1600 containing processor 103 configured to be placed at a temporal artery of an ear. In one example, the BPM component 1600 may incorporate an ear bud 1602 that is inserted into the ear canal. In another example, as illustrated in FIG. 16C-FIG. 16D, the BPM component 1600 may incorporate a clip 1604 that attaches to the tragus of the ear to prevent movement of the device while the user is in movement. The ear bud 1602 or ear clip 1604 is supported by a rigid backing 1614. The rigid backing also houses suitable electronics 1616. The ear clips 1604 adjust in size, as illustrated by arrows 1606, in order to fit securely on ears of various sizes. The BPM component 1600 incorporates a pressure sensor 1608 with a protective layer 1610 for acquiring an arterial waveform. In one example, the BPM component 1600 further incorporates a temperature sensor (not shown) inside the ear bud 1602 or ear clips 1604 for acquiring the temperature of a patient's body and an ambient temperature sensor 2100.

In one example, a wearable measurement device includes four independent components, a BPM 1600, temperature sensor for ambient temperature 2100 and a temperature sensor for measuring the patient's body temperature 1602 and a Blood oxygen saturation (BOS)) 1700 that can be used in combination or independently of one another. The BPM 1600, a BOS 1700, an ambient temperature 2100 sensor and a body temperature sensor 1602 each may include one or more sensors and their own electronics or power source. The BPM 1600, a BOS 1700, an ambient temperature 2100 sensor and a body temperature sensor 1602 gather information independently but transfer the information to the same patient computing device.

Using FIG. 16 E and FIG. 16F the BPM component 1600 further includes a rigid arm 1612 for providing structure and support between the rigid backing 1614 of the ear bud 1602 or ear clips 1604 and the pressure sensor 1608 with protective layer 1610. The rigid arm 1612 is composed of two pieces first piece 2205 for receiving extension piece 2200. The extension piece 2200 is slideably adjustable with respect to the first piece 2205 so as to permit the patient to align the BPM component 1600 with the temporal artery 514. The rigid arm 1612 further provides structure support for the pressure sensor 1608 with protective layer 1610 while the rigid backing 1614 provides structure support for the ear clips 1604 and the ear bud 1602 which incorporates the optical sensor (not shown). The arm is designed with a hinge or adjustable property to allow for flexibility 1614 and proper positioning of the BPM component 1602 to the temporal artery 514.

FIG. 17A and FIG. 17B illustrates an example BOS component 1700 of a wearable measurement device configured to be placed at the lobe or pinna of the ear. The BOS component includes LEDS 1702 and a photodiode 1704 for measuring absorbed light. In one example, the BOS component 1700 is designed in the form of a clip that allows the photodiode 1704 to be placed on one side of the ear and the LEDs 1702 to be placed on the opposing side. The clip will clamp down <along hinge 1706 on the lobe or pinna of the ear to ensure movement of the device is reduced to a minimum. In one example, a portion of the BOS component 1700 will have the ability to move to allow safe and quick removal of the clip. The BOS component 1700 includes a clip housing 1710 for providing support and structure for the LEDS 1702 and the photodiode 1704. Suitable electronics 1708 are disposed inside the clip housing 1710.

In one example, the BPM 1600, ambient temperature sensor 2100 and BOS 1700 components each include a power source (not shown). In another example, the BPM 1600, ambient temperature sensor 2100 and the BOS 1700 components share a power source 1690. For example, the BPM component 1600 and ambient temperature sensor 2100 may contain a power source while the BOS 1700 component may couple to the BPM component 1600 in order for power to transfer to the BOS component 1700. The power source 1690 can be selected from the group consisting of a battery a power supply or a solar collector.

In one example, both the BPM component 1600, ambient temperature sensor 2100 and the BOS component 1700 can be worn simultaneously at the ear. In another example, only one of the BPM component 1600, ambient temperature sensor 2100 and the BOS component 1700 may be worn as the patient desires.

As shown in FIG. 20A, and FIG. 20B the wearable measurement device 102 may be selectively positioned on any portion of the patient's body 104 suitable for continuously monitoring blood pressure from the superficial temporal artery 514 by rotating the arm 112 about pivot 512 such that it aligns with temporal artery 514 of the head 114. The spring 520 provides an upward force on arm 112 such that wearable measurement device 102 is pressed to insure that the wearable measurement device 102 is in communication with temporal artery 514 of the head 114. This force provided by spring 520 allows the wearable measurement device 102 to automatically adjust for differing facial structures of head 114. The ability to rotate the arm 112 about pivot 512 allows the patient or healthcare provider to align with temporal artery 514 of the head 114 and adjust for differing facial structures of head 114.

The example wearable measurement devices described herein incorporate advances in battery technology, RF-powering, and energy storage techniques. In order to power the sensors and discrete components while consuming a low amount of power, the wearable measurement devices includes a custom integrated circuit component that will greatly reduce the size, complexity, and the power consumption. The circuit can be designed in a suitable way to accommodate signal processing and control of the wearable measurement devices.

In one example, a battery is utilized to power the wearable measurement device's components. The battery will provide power for wireless signal transmission to a patient computing device and for the discrete components. In one example, the battery is replaceable or rechargeable. Rechargeable power sources can be charged through a wired connection such as a direct plug-in through a wall outlet or through micro-USB charging where the ear cuff contains the female end of the micro-USB plug. In one example, the wearable measurement device includes energy harvesters to acquire and store energy. Energy harvesters such as those that harvest energy from heat, sunlight, or vibration may be used. This may ensure a longer time of use for the patient. In one example, the wearable measurement device can be charged wirelessly. To ensure proper operation of the wearable measurement device, common power regulating circuits will be used to maximize efficiency and longevity of use. In another example, the wearable measurement device can be wirelessly charged through inductively coupled circuits. No battery is needed in this particular example, but proper regulation of the acquired energy is provided by power electronic circuitry.

It should be appreciated that data transmission between a wearable measurement device and a patient computing device will be done wirelessly through suitable technologies and protocols such as Bluetooth, Zigbee or Near Field Comunication (NFC) and other short range data transmission techniques. In one example, proper conversion of the signals contained must be performed to allow efficient transfer of the information. As an example, the sensors may output an analog signal which will need to be converted to a digital signal before being wirelessly transferred to the patient computing device. In one example, Bluetooth technology may be used which is low in cost, easy to interface, small in size, and requires low power operation. In one example, near field communications (“NFC”) can be used for transmitting data to the patient computing device. NFC technology utilizes small circuit components and is low power. With NFC technology, the patient can swipe or move the patient computing device into proximity of the site of the BPM or BOV to initiate transmission of the information. In one example, radio-frequency identification (RFID) can be used for transmitting data to the patient computing device. The signals acquired by the patient computing device are translated and displayed onto a user interface.

It should be further appreciated that, although wireless communication is described herein, the wearable measurement device may further be configured to communicate data to the patient computing device via wired connection. For example, the wearable measurement device may include a data port, such as a USB port, to facilitate communication with a patient computing device. In one example, either the same port or an additional port may be used to facilitate charging the battery of the wearable measurement device.

In one example, a wearable measurement device includes the ability to track the amount of steps and the posture of the patient. By incorporating micro-electric-mechanical systems (“MEMS”), including accelerometers and gyroscopes, into a wearable measurement device, data indicative of the position of the patient, the number of steps taken, whether the patient is exercising, and for how long the patient is exercising can be captured.

In one example, the wearable measurement device has the ability to track the period of use and when the patient uses it. For example, when a blood pressure waveform is acquired and detected, a timer is initiated that will count the number of seconds of use. In another example, the wearable measurement device can use the MEMS devices to know when the device is worn through vibration characteristics. This information can be displayed on the interface of the patient computing device. In one example, the wearable measurement device sends reminders in the form of audio or visual alerts through the user interface of the patient computing device when the device has been inactive or unused for a certain time. Tracking of such information may be useful for ensuring compliance, for example.

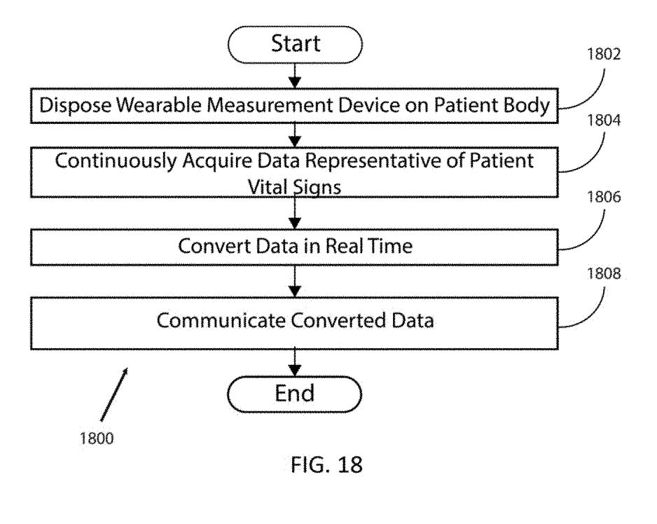

FIG. 18 illustrates an example method 1800 for measuring patient vital signs. At step 1802, a wearable measurement device is disposed on a patient body using a suitable mechanism such as a clip, a strap, adhesive, and so on. In one example, the wearable device is disposed on a patient's ear. In one example, a plurality of wearable measurement devices are disposed on the patient's body.

At step 1804, the wearable measurement device continuously obtains data representative of the patient's vital signs. For example, the wearable measurement device continuously obtains data such as blood pressure, blood oxygen saturation, heart rate, body temperature, respiratory rate, and body position. In one example, the wearable measurement device stores the obtained data, while in another example, the wearable measurement device communicates the obtained data to a third-party computing device.

At step 1806, the obtained data is converted and formatted. For example, the obtained data may be converted into a format that is more easily interpreted by a user and more meaningful for the user. In one example, the obtained data is converted by the wearable measurement device. In another example, the data is converted by a third-party computing device.

At step 1808, the converted data is presented to a user. In one example, the data is presented to the user at the wearable communication device. In one example, the data is presented to a user, such as a patient, a doctor, a family member, or another suitable party, via a third-party computing device. In one example, the converted data is first communicated to the wearable measurement device by the third-party computing device before the wearable computing device presents the data. Data presented to the user may include, for example, systolic blood pressure measured in mmHg, diastolic blood pressure measured in mmHg, blood oxygen saturation measured in percentage, heart rate measured in beats per minute, respiratory rate measured in breaths per minute, body temperature measured in degrees Fahrenheit or degrees Celsius. Displayed information can further include signals such as an arterial waveform, polyplethysmography, and respiratory rate.

In one example, the third-party computing device stores the received and converted data in a data store associated with the patient from which the vital sign data was obtained. For example, the data may be stored in an EMR record associated with the patient.

It should be appreciate that a patient, as referenced throughout the description herein, may include a human or any suitable animal for which it may be desirable to collect vital sign data.

It should be appreciated that the third-party computing device 110 of FIG. 1, including a user interface, may be any suitable form such as a smart watch, an electronic display, a mobile application on a smart phone, tablet, or any other smart device, or an application on any personal computing device. The user interface of the patent computing device may contain information such as the patient's name, medical condition (if any), current medications, age, the number of calories burned, the number of steps taken, weight, usage time, blood pressure, blood oxygen saturation, heart rate, or body temperature. Physicians, family, friends, or other third parties can also be issued a user interface via a third-party computing device and be given access to patient information or be alerted when vital signs are outside of a normal range of the patient. Additionally, the user interface will contain software to translate the signals acquired from the wearable measurement devices. Thus, the wearable measurement device is solely designed to acquire the signal while the user interface portion of the patient computing device is designed to translate the signals into meaningful data such as blood pressure or heart rate. In one example, the wearable measurement device may be configured to translate or manipulate the acquired data before transmitting the data to the patient computing device.

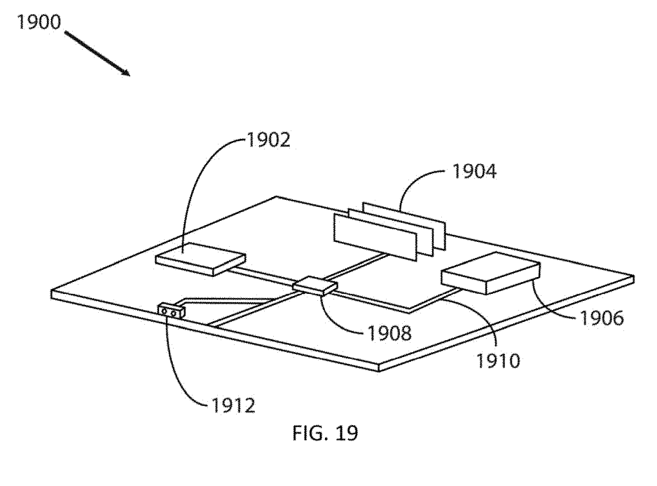

FIG. 19 is a schematic diagram of an example computer 1900 for implementing the example third-party computing device 110 of FIG. 1. Computer 1900 includes a processor 1902, memory 1904, a storage device 1906, and a communication port 1908 operably connected by an interface 1910 via a bus 1912. Processor 1902 processes instructions, via memory 1904, for execution within computer 1900. In an example embodiment, multiple processors along with multiple memories may be used.

Memory 1904 may be volatile memory or non-volatile memory. Memory 1904 may be a computer-readable medium, such as a magnetic disk or optical disk, solid-state drive (SSD), Random Access Memory, disk drive or tape drive device. Storage device 1906 may be a computer-readable medium, such as floppy disk devices, a hard disk device, optical disk device, a tape device, a flash memory, phase change memory, or other similar solid state memory device, or an array of devices, including devices in a storage area network of other configurations. In one example, the storage device 1906 includes dual solid state disk drives. A computer program product can be tangibly embodied in a computer-readable medium such as memory 1904 or storage device 1906.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use, See, Bryan A. Garner, A Dictionary of Modern Legal Usage 624 (2d. Ed. 1995). Also, to the extent that the terms “in” or “into” are used in the specification or the claims, it is intended to additionally mean or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed:

1. A portable wearable computing device configured in the form of an ear bud to continuously obtain data indicative of a patient's vital signs from the region associated with a patient's ear, the portable wearable computing device comprising:
A first processor,
A first temperature sensor configured to obtain data indicative of body temperature of the patient from the tympanic membrane,
Said first temperature sensor rotatably mounted from the hub to align with a patient's ear canal,
Said hub configured to push said first temperature sensor into the patient's ear canal,
a second temperature sensor configured to obtain data indicative of ambient temperature of the patient environment,
blood oxygen saturation sensor configured to obtain data indicative of amount of oxygen present in the patient's blood from the patient's ear lobe,
an arterial waveform sensor configured to obtain data indicative of an arterial waveform produced by the patient's superficial temporal artery,
Said arterial waveform sensor being connected to an arm that pivots about said ear bud such that it rotates radially about said ear,
Said arm having a spring proximal to said ear bud such that said spring rotates said waveform sensor to apply pressure to said patient's superficial temporal artery,
a second processor having a display and power source coupled to it,
Said first processor coupled to the ambient temperature sensor, body temperature sensor, the blood oxygen sensor, and the blood pressure sensor, and configured to receive the obtained data indicative of the patient's vital signs comprising of data from said ambient temperature sensor, said body temperature sensor, said blood oxygen sensor, and said blood pressure sensor and said first processor wirelessly coupled to said second processor,
Said second processor containing an algorithm to modify the arterial waveform sensor data by reducing the pressure readings by X% for ambient temperatures above 34 degrees C./93 degrees and increasing the pressure readings by Y% when the ambient temperature was below 28 degrees C./82 degrees.

2. The portable wearable computing device of claim 1, where X is a value between—−0.02% to −0.1%.

3. The portable wearable computing device of claim 1, where Y is a value between +0.03% to +0.1%.

4. The portable wearable computing device of claim 1, further comprising a computer readable tangible storage device, wherein the said second processor is further configured to store the received data indicative of the patients vital signs in the computer readable tangible storage device.

5. The portable wearable computing device of claim 1, wherein the processor is further configured to derive the systolic and diastolic blood pressure based on the received data indicative of the arterial waveform and the ambient temperature.

6. The portable wearable computing device of claim 1, further comprising a wireless antenna, wherein the processor is further configured to communicate the received data indicative of the patient's vital signs via the wireless antenna.

7. The portable wearable computing device of claim 1, wherein arterial waveform sensor comprises a pressure sensor and a flexible protective layer disposed over the sensor, and wherein the pressure sensor in combination with the flexible protective layer are configured to detect vibrations exhibited from arterial palpitation by the patient.

8. The portable wearable computing device of claim 1, wherein the blood oxygen saturation sensor comprises an LED light source configured to emit light and a light sensor configured to measure the amount of emitted light absorbed by the patient.

9. The portable wearable computing device of claim 1, further comprising an ear clip configured to secure device to an ear of the patient by clipping to the helix of the ear.

10. The portable wearable computing device of claim 1, further comprising an accelerometer and a gyroscope for measuring the patient's body position.

11. A method for continuously obtaining vital sign data, comprising the step of:
disposing a wearable measurement device on a patient's body,
Said wearable measurement device having a first processor configured to receive data relative to a patient's vital signs from a pressure sensor, pulse oximetry sensor, temperature sensor and ambient temperature sensor,
Said fire processor continuously acquiring data from said wearable measurement device pressure sensor, pulse oximetry sensor and temperature sensor
measuring the ambient temperature of the patient's environment,
Said first processor configured to send data wirelessly from said wearable measurement device pressure sensor, pulse oximetry sensor and temperature sensor to a second processor on a remote computer,
Said first processor converting the acquired data in real time based on the data from the pulse oximetry sensor and temperature sensor and the data from the ambient temperature sensor,
Said first processor using the said ambient temperature sensor data and converting the acquired data in real time based on the data from the pressure sensor and said ambient temperature sensor data such that said first processor contains an algorithm to modify the arterial waveform sensor data by reducing the pressure readings by X% for ambient temperatures above 34 degrees C./93 degrees and increasing the pressure readings by Y% when the ambient temperature was below 28 degrees C./82 degrees; and communicating the converted data to a second processor on a remote computer.

12. The portable wearable computing device of claim 1, where X is a value between—−0.02% to −0.1%.

13. The portable wearable computing device of claim 1, where Y is a value between +0.03% to +0.1%.

14. The method of claim 11, wherein the step of converting the acquired data in real time comprises deriving systolic and diastolic blood pressure based on the received data representative of the arterial waveform.

15. The method of claim 11, wherein the step of communicating the converted data comprises communicating, the converted data to a display.

16. The method of claim 11, wherein the step of communicating the converted data comprises communicating the converted data to an electronic medical records database.

17. The method of claim 11, wherein the step of disposing the wearable measurement device on the patient's body comprises disposing the wearable measurement device on the patient's ear.

18. The method of claim 11, further comprising the step of disposing a plurality of wearable measurement devices on the patient's body and continuously acquiring data representative of the patient's vital signs from the plurality of wearable measurement devices.

19. A non-invasive system for continuously monitoring blood pressure of a patient, the system comprising:
a sensor disposed on the patient, the sensor configured to acquire data indicative of an arterial waveform from the patient and communicate it to a first processor and said first processor in communication with an ambient temperature sensor and to wirelessly communicate the acquired data indicative of the said arterial waveform and said ambient temperature to a patient computer,
Said patient computer configured to receive the communicated data indicative of the said arterial waveform,
Said patient computer using the said ambient temperature sensor data and converting the acquired data in real time based on the data from the pressure sensor and said ambient temperature sensor data to systolic and diastolic blood pressure data; and communicating the converted data to a second processor on a remote computer.

20. The system of claim 19, wherein the patient computer is further configured to communicate the systolic and diastolic blood pressure to one of a display monitor and a patient electronic medical record.
